(19) United States
(12) Patent Application Publication
(10) Pub. No.: US 2025/0352814 A1
(43) Pub. Date: Nov. 20, 2025

(54) SYSTEM AND METHOD FOR MAGNETIC NANOPARTICLE THERMAL ABLATION

(71) Applicant: BARD PERIPHERAL VASCULAR, INC., Franklin Lakes, NJ (US)

(72) Inventors: Arieona BOYLE, Phoenix, AZ (US); Elizabeth BURKETT, Scottsdale, AZ (US); Lindsay DONNELLY, Chandler, AZ (US); Preston FRANKLIN, Mesa, AZ (US); Jacob N. GILSON, Phoenix, AZ (US); Matt H. KAS, Chandler, AZ (US); Heather A. STORM, Phoenix, AZ (US); Cassandra VAN ALLEN, San Tan Valley, AZ (US)

(73) Assignee: BARD PERIPHERAL VASCULAR, INC., Franklin Lakes, NJ (US)

(21) Appl. No.: 18/867,713

(22) PCT Filed: Jun. 15, 2022
(86) PCT No.: PCT/US2022/033559

§ 371 Date: Nov. 20, 2024

(51) Int. Cl. A61N 2/00; A61N 2/02

(52) U.S. Cl. CPC A61N 2/004; A61N 2/002; A61N 2/02

(57) ABSTRACT

Systems and methods for thermally ablating diseased tissue a patient includes a plurality of magnetic nanoparticles for disposal at a target site, a retention element configured to be attached to the patient proximate to the diseased tissue, an inductive magnetic field generator element disposed within the retention element, and a signal generator coupled to the inductive magnetic field generator element and configured to generate an electric signal. The inductive magnetic field generator element is configured to generate an alternating magnetic field based on the electrical signal, and the alternating magnetic field is configured to interact with the plurality of magnetic nanoparticles at the target site to induce hysteresis and thereby increase a temperature of the plurality of magnetic nanoparticles to a desired temperature to ablate the diseased tissue.

### TECHNICAL FIELD

The present disclosure relates to systems and methods of using magnetic nanoparticles, and more particularly, to a system and method of using a plurality of magnetic nanoparticles for thermal ablation of cancerous tissues.

### BACKGROUND

Invasive and non-specific therapies, such as surgery, percutaneous ablation, radiation, and chemotherapy, may be employed in treating cancers. Often, multiple treatments are required to reach all margins of a tumor. Such cancer treatments may have several limitations, including, but not limited to, the destruction of healthy tissue, long recovery times, and complications from bleeding and infection.

Accordingly, a need exists for minimally invasive treatments of cancerous tissues that address the above-noted limitations of current treatment modalities.

### SUMMARY

According to an embodiment of the present disclosure, a system for thermally ablating a diseased tissue at a target site in a patient includes a plurality of magnetic nanoparticles for disposal at the target site, a retention element configured to be attached to the patient proximate to the diseased tissue, an inductive magnetic field generator element disposed within the retention element, and a signal generator coupled to the inductive magnetic field generator element. The signal generator is configured to generate an electric signal. The inductive magnetic field generator element is configured to generate an alternating magnetic field based on the electrical signal, and the alternating magnetic field is configured to interact with the plurality of magnetic nanoparticles at the target site to induce hysteresis increase and thereby a temperature of the plurality of magnetic nanoparticles to a desired temperature to ablate the diseased tissue.

In another embodiment, a system for thermally ablating diseased tissue a patient includes a plurality of magnetic nanoparticles for disposal at the target site, an inductive magnetic field generator element disposed proximate to the diseased tissue, a signal generator coupled to the inductive magnetic field generator element, and a detector configured to monitor the alternating magnetic field. The detector is configured to be coupled to the inductive magnetic field generator element. The signal generator is configured to generate an electric signal, the inductive magnetic field generator element is configured to generate an alternating magnetic field based on the electrical signal, and the alternating magnetic field is configured to interact with the plurality of magnetic nanoparticles to induce hysteresis and thereby increase a temperature of the plurality of magnetic nanoparticles to a desired temperature to ablate the diseased tissue.

In another embodiment, a method for thermally ablating diseased tissue at a target site in a patient includes delivering a plurality of magnetic nanoparticles to the diseased tissue at the target site, attaching a retention element including an inductive magnetic field generator element disposed therein to a skin of the patient proximate to the diseased tissue, generating an electrical signal using a signal generator coupled to the inductive magnetic field generator element, and generating an alternating magnetic field using the inductive magnetic field generator element and the electrical signal. The alternating magnetic field interacts with the plurality of magnetic nanoparticles to induce hysteresis. The method further includes increasing a temperature of the plurality of magnetic nanoparticles based on the induced hysteresis to a desired temperature to ablate the diseased tissue.

These and additional features provided by the embodiments described herein will be more fully understood in view of the following detailed description, in conjunction with the drawings.

### BRIEF DESCRIPTION OF DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is a cut-away perspective view of a system for thermally ablating diseased tissue a patient, according to one or more embodiments shown and described herein;

FIG. 2 schematically depicts the system of FIG. 1 including control modules for implementing computer and software based methods for thermally ablating diseased tissue in a patient, according to one or more embodiments shown and described herein;

FIG. 3 schematically depicts a partial cut-away perspective view of a magnetic nanoparticle of the system of FIG. 1, according to one or more embodiments shown and described herein;

FIG. 4 is a perspective view of a retention element of the system of FIG. 1, according to one or more embodiments shown and described herein;

FIG. 5 is a schematic view of the delivery of the plurality of magnetic nanoparticles into the vessels, according to one or more embodiments shown and described herein;

FIG. 6 is a schematic view of the interaction of the alternating magnetic field with the plurality of magnetic nanoparticles in the vessels, according to one or more embodiments shown and described herein; and

FIG. 7 depicts a flowchart of an exemplary method for thermally ablating diseased tissue a patient, according to one or more embodiments shown and described herein.

Corresponding reference characters indicate corresponding parts throughout the several views. The exemplifications set out herein illustrate at least one embodiment of the invention, and such exemplifications are not to be construed as limiting the scope of the invention in any manner.

### DESCRIPTION

Embodiments of the systems and methods of the present disclosure include magnetic nanoparticles that work in conjunction with a magnetic field generator and a transdermal patch to provide localized hyperthermia and ablate all margins of a tumor while preserving healthy tissue. Using the system or method of the present disclosure permits a single treatment to ablate and size, shape, or stage of tumor. Further, following the method disclosed herein may reduce recovery time and complications when treating cancer compared to other cancer treatments. The systems and methods disclosed herein may provide for real-time visualization of the magnetic nanoparticles and a single, catheter-based infusion for controlled delivery. The magnetic field generator described herein may provide focused, accurate, and controlled hyperthermal ablation of cancerous cells to meet the needs of individual patients. The systems and methods disclosed herein further provide for a controlled determination and application of each of an intensity, a treatment duration, and a frequency of an induced alternating magnetic field as controlled by an electrical signal to interact with a plurality of magnetic nanoparticles determined based on a combination of an estimated concentration of the plurality of magnetic nanoparticles present within the diseased tissue and a specific absorption rate associated with a type of material and size of the plurality of magnetic nanoparticles delivered to the diseased tissue. The electrical signal may be applied as an alternating electrical current to cooperate with an inductive magnetic field generator element such as a copper wire coil to cause a change in magnetic flux and induce an alternating electromagnetic field (also referenced herein as alternating magnetic field), which field may change based on a change in the applied electrical signal. In embodiments, a direct current may be utilized via the electrical signal to induce the magnetic field, which may alternate based on alternating changes in the direct current (i.e., being decreased then increased, being increased the decreased, or being turned on and off). The alternating magnetic field further induces hysteresis, which is a dynamic lag of magnetic induction behind the magnetizing force, and in which work done by the magnetizing force against internal friction of, for example, the magnetic nanoparticles produces heat as hysteresis loss.

In effect, embodiments described herein are generally directed to systems and methods for thermally ablating diseased tissue, e.g., liver abnormalities caused by hepatic cancer, in a patient. The systems and methods include at least a delivery probe configured to deliver a plurality of magnetic nanoparticles to a target site having diseased tissue, a retention element configured to be attached to the patient proximate to the target site, an inductive magnetic field generator element disposed within the retention element, and a signal generator coupled to the inductive magnetic field generator element. The systems and methods disclosed herein may provide for a single. minimally invasive treatment that results in the ablation of all margins of a cancerous tumor, no matter the size, shape, or stage of the tumor, while also preserving healthy tissue. The visualization of the magnetic nanoparticles in combination with other factors, such as distance between the retention element and the target site, may allow the user of the system to provide focused, accurate, and controlled hyperthermal ablation of cancerous cells to meet the needs of individual patients. As described in greater detail further below, the plurality of magnetic nanoparticles may include therapeutic agents or radioactive materials to provide a combination of treatments.

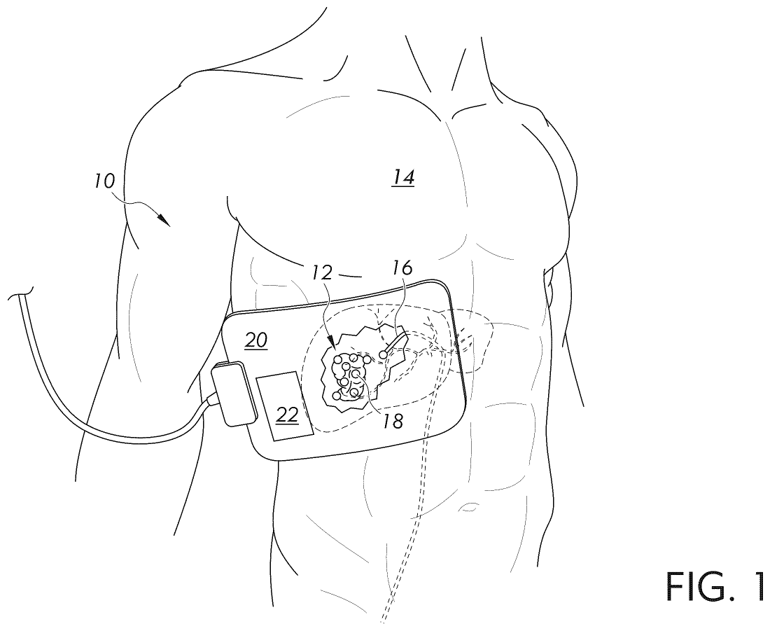

Referring now to the drawings, and more particularly to FIG. 1, an embodiment of a system 10 for thermally ablating diseased tissue at a target site 12 in a patient 14 is provided. A delivery probe 16 and a plurality of magnetic nanoparticles 18 for disposal at the target site 12 are shown in FIG. 1. The delivery probe 16 is configured to deliver the plurality of magnetic nanoparticles 18 to the target site 12 inside the patient 14. The system 10 also includes a retention clement 20 that is configured to be attached to the patient 14 proximate the target site 12 containing and thus proximate to the diseased tissue. An inductive magnetic field generator element 22 is disposed within the retention element 20. In embodiments, the system 10 includes the inductive magnetic field generator element 22 disposed proximate to the diseased tissue (i.e., not included in the retention element 20). In such embodiments, the inductive magnetic field generator element 22 may include a material or covering feature to protect the skin of the patient to which it may be adhered proximate the diseased tissue.

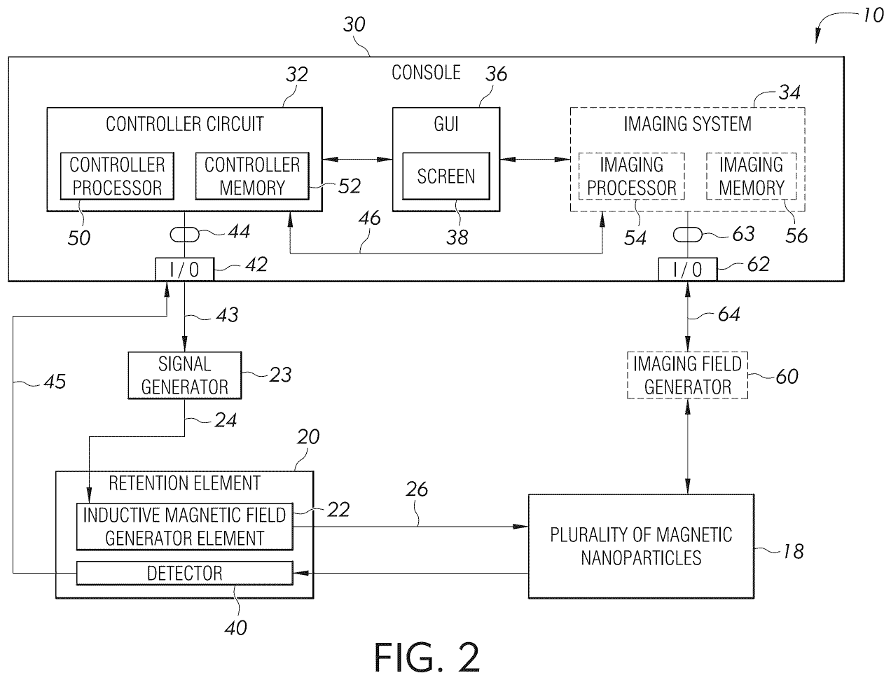

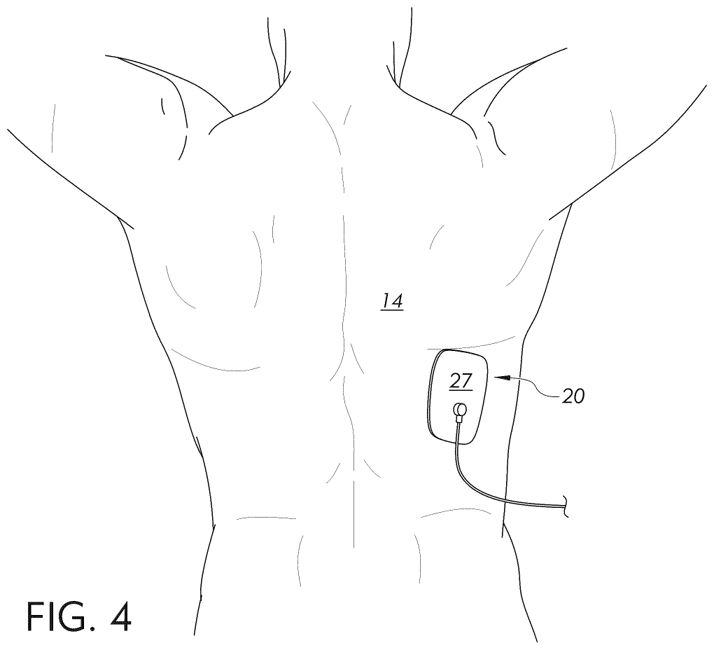

Referring to FIG. 2, a signal generator 23 is coupled to the inductive magnetic field generator element 22 (such as via a wire shown in FIG. 4). The signal generator 23 is configured to generate an electrical signal 24 (FIG. 2), such as an alternating current. Receiving the electrical signal 24, the inductive magnetic field generator element 22 is configured to generate an alternating magnetic field 26 based on the electrical signal 24. The alternating magnetic field 26 is configured to interact with the plurality of magnetic nanoparticles 18 at the target site 12 to induce hysteresis and thereby increase a temperature of the plurality of magnetic nanoparticles 18 and, consequently, the diseased tissue at the target site 12 to a level in which the diseased tissue is ablated (e.g., increase the temperature of the plurality of magnetic nanoparticles 18 to a desired temperature to ablate the diseased tissue).

In some embodiments, the retention element 20 includes a transdermal patch 27, as shown in FIG. 4, which includes an adhesive layer that is configured to adhere to the patient 14 proximate to the diseased tissue. The retention element 20 is configured to be placed proximate the target site 12 having the diseased tissue. The system 10 may provide for the alternating magnetic field 26 to be configured to increase the temperature of the plurality of magnetic nanoparticles 18 to a desired temperature of greater than or equal to 60° C., resulting in the necrosis of the diseased tissue at the target site 12. Thus, the alternating magnetic field may be configured to increase the temperature of the plurality of magnetic nanoparticles 18 to the desired temperature of greater than or equal to 60° C. to ablate the diseased tissue.

Referring again to the block diagram of FIG. 2, the system 10 may include one or more control modules beyond the signal generator 23, such as a console 30 having a controller circuit 32. In some embodiments, the system 10 includes an imaging system 34 and a graphical user interface (i.e., GUI) 36 communicatively coupled to the console 30. The GUI 36 may include a screen 38, which may be a display screen or a touch-screen configured to accommodate user input. The controller circuit 32 may be communicatively coupled to and thus in communication with the signal generator 23 via a retention element input/output (I/O) interface circuit 42, a first internal bus structure 44, and a first communication cable 43. The controller circuit 32 may be communicatively coupled to the retention element 20, which may include a detector 40, via the retention element I/O interface circuit 42, the first internal bus structure 44, a second communication cable 45, and the signal generator 23. The detector 40 may be configured to monitor the alternating magnetic field 26, and the detector 40 may be configured to be coupled to the inductive magnetic field generator element 22.

The controller circuit 32 is an electrical circuit that has data processing capability and command generating capability. In the present embodiment, the controller circuit 32 has a controller processor 50 and a controller memory 52, which is an associated non-transitory electronic memory. The controller processor 50 may be in the form of a single microprocessor, or two or more parallel microprocessors. The controller memory 52 may include multiple types of digital data memory, such as random access memory (RAM), non-volatile RAM (NVRAM), read only memory (ROM), and/or electrically erasable programmable read-only memory (EEPROM). The controller memory 52 may further include mass data storage in one or more of the electronic memory forms described above, or on a computer hard drive or optical disk. Alternatively. controller circuit 32 may be assembled as one or more Application Specific Integrated Circuits (ASIC).

The controller memory 52 may store data on specific absorption rates of the plurality of magnetic nanoparticles 18 associated with any of the following factors: (1) an identified material composition of the plurality of magnetic nanoparticles 18; (2) a known average size of the plurality of magnetic nanoparticles 18; (3) an estimated concentration of the plurality of magnetic nanoparticles 18; (4) a temperature of the plurality of magnetic nanoparticles 18; (5) a distance between the plurality of magnetic nanoparticles 18 and an inductive magnetic field generator element 22; and (6) any of the preceding in combination. The controller processor 50 may be configured to execute program instructions stored in the controller memory 52 to execute one or more control schemes described herein, such as to use the data on the specific absorption rates, as described above, to calculate a calculated specific absorption rate of the plurality of magnetic nanoparticles 18 having at least one selected from the group of an identified material composition of the plurality of magnetic nanoparticles 18, a known average size of the plurality of magnetic nanoparticles 18, an estimated concentration of the plurality of magnetic nanoparticles 18, a temperature of the plurality of magnetic nanoparticles 18, a known distance between the plurality of magnetic nanoparticles 18 and the inductive magnetic field generator element 22 or any combination of these factors.

In addition, the controller memory 52 may store data on the effect of (1) frequencies of an alternating magnetic field 26 interacting with a plurality of magnetic nanoparticles 18; (2) intensities of current level data associated with alternating magnetic field 26 interacting with a plurality of a magnetic nanoparticles 18; (3) treatment durations of the alternating magnetic field 26 interacting with the plurality of magnetic nanoparticles 18; or (4) any of the preceding in combination, on the temperature of the plurality of magnetic nanoparticles 18 having a calculated specific absorption rate. The controller processor 50 may be configured to execute program instructions, which are received from a program source, such as software or firmware, to which the controller circuit 32 has electronic access, and/or stored in the controller memory 52 to process the calculated specific absorption rate of the plurality of magnetic nanoparticles 18 in order to select a frequency, an intensity, and a treatment duration of the induced magnetic field 26 as controlled by the electrical signal 24 delivered to the inductive magnetic field generator element 22 to generate the alternating magnetic field 26 to cooperate with the plurality of magnetic nanoparticles 18 to produce a desired temperature of the plurality of magnetic nanoparticles 18 via a temperature increase caused by a resulting induced hysteresis.

In some embodiments, the controller circuit 32 is communicatively coupled to the imaging system 34 via a second internal bus structure 46. In the embodiments in which the system 10 includes the imaging system 34, the imaging system 34 is configured to generate imaging data that contains a visualization of the plurality of magnetic nanoparticles 18. In embodiments, the controller circuit 32 is in communication with the signal generator 23, the controller circuit 32 being configured to estimate a concentration of the plurality of magnetic nanoparticles 18 within the diseased tissue and identify a specific absorption rate (SAR) of the plurality of magnetic nanoparticles 18. The controller circuit 32 may be is configured to determine an intensity of the alternating magnetic field 26, a frequency of the alternating magnetic field 26, and a treatment duration to apply based on the estimated concentration of the plurality of magnetic nanoparticles 18 within the diseased tissue and the SAR of the plurality of magnetic nanoparticles 18. In embodiments, the controller circuit 32 is in communication with the signal generator 23 and the imaging system 34, the controller circuit 32 being configured to estimate a concentration of the plurality of magnetic nanoparticles 18 within the diseased tissue based on the image from the imaging system 34 and identify a specific absorption rate (SAR) of the plurality of magnetic nanoparticles 18. The imaging system 34 is an electrical circuit that has data processing capability and command generating capability. The imaging memory 56 is an associated non-transitory electronic memory. The imaging processor 54 may be in the form of a single microprocessor, or two or more parallel microprocessors. The imaging memory 56 may include multiple types of digital data memory, such as random access memory (RAM), non-volatile RAM (NVRAM), read only memory (ROM), and/or electrically erasable programmable read-only memory (EEPROM). The imaging memory 56 may further include mass data storage in one or more of the electronic memory forms described above, or on a computer hard drive or optical disk. Alternatively. imaging system 34 may be assembled as one or more Application Specific Integrated Circuits (ASIC).

In embodiments, the controller circuit 32 receives detected data from the detector 40 to measure the intensity and frequency of the alternating magnetic field 26 being applied externally from the retention element 20 to the plurality of magnetic nanoparticles 18. The detector 40 may be configured to monitor the electrical signal 24 generated by the signal generator 23 that cooperates with the inductive magnetic field generator element 22 to generate the induced alternating magnetic field 26. As the detector 40 monitors the electrical signal 24, the detector 40 is configured to produce current level data associated with the electrical signal 24. The detector 40 may be attached to the retention element and in communication with the controller circuit, and the detector 40 may be configured to deliver the current level data to the controller circuit 32. The controller circuit 32 may be configured to determine and apply each of an intensity, a treatment duration, and a frequency of the alternating magnetic field 26 to be delivered to the plurality of magnetic nanoparticles 18 based on the concentration of the plurality of magnetic nanoparticles within the diseased tissue and the specific absorption rate of the plurality of magnetic nanoparticles 18. The controller circuit 32 may be configured to calculate the magnitude of the alternating magnetic field 26 based on the current level data received from the detector 40. Additionally or alternatively, the detector 40 may be configured to monitor the temperature of the target site 12 and produce temperature data to deliver to the controller circuit 32.

In still other embodiments, the controller processor 50 may be configured to estimate a concentration of the plurality of magnetic nanoparticles 18 within the target site 12, which contains the diseased tissue, based on processing the imaging data produced by the imaging system 34. The imaging system 34, if present, may include an imaging processor 54 and an imaging memory 56. The imaging system 34 may be communicatively coupled to an imaging field generator 60. The imaging system 34 may be communicatively coupled to the imaging field generator 60 via an imaging field generator input/output (I/O) interface circuit 62, a third internal bus structure 63, and a third communication cable 64. In some embodiments, the imaging field generator 60 may be located in an ultrasound probe configured to produce an ultrasound field-of-view volume. However, in still other embodiments, the imaging field generator 60 may be an X-ray device or another known imaging modality for providing accurate imaging data. The imaging field generator 60 may be internal or external to the console 30. In embodiments, the imaging field generator 60 may be located within the retention element 20. The imaging system 34 may be configured to produce the imaging data concerning the concentration of the plurality of magnetic nanoparticles 18 being delivered or already delivered to the target site 12.

The controller memory 52 may include tables of data on the specific absorption rate (SAR) of the plurality of magnetic nanoparticles 18 that are produced at various concentrations of the plurality of magnetic nanoparticles 18, distances of the plurality of magnetic nanoparticles 18 from the inductive magnetic field generator element 22, and the various qualities of the alternating magnetic field 26 (magnitude, frequency, and length of treatment duration). The controller processor 50 accesses these tables of data in order to determine parameters of the electrical signal 24 (e.g., current, duty cycle, frequency, and/or amplitude) to be delivered to the retention element 20 and to select the treatment duration for the alternating magnetic field 26 to produce a selected specific absorption rate (SAR) of the plurality of magnetic nanoparticles 18. The controller processor 50 may be configured to execute program instructions, which are received from a program source, such as software or firmware, to which the controller circuit 32 has electronic access, and/or stored in the controller memory 52 to identify the specific absorption rate of the plurality of magnetic nanoparticles 18. With the controller circuit 32 having data on the size of the target site 12, data on the concentration and location of the plurality of magnetic nanoparticles 18, tables of data on the specific absorption rate (SAR) of the plurality of magnetic nanoparticles 18, and the controller circuit 32 being configured to control the production of alternating magnetic field 26 delivered to the plurality of magnetic nanoparticles 18 at the target site 12, the controller circuit 32 is configured to provide focused, accurate, and controlled hyperthermal ablation of cancerous cells at the target site 12 to meet the needs of the patient 14.

As described above, data on the concentration of the plurality of magnetic nanoparticles 18 present at the target site 12 may be produced by the imaging system 34 or based on the delivery of the plurality of magnetic nanoparticles 18 from the delivery probe 16.

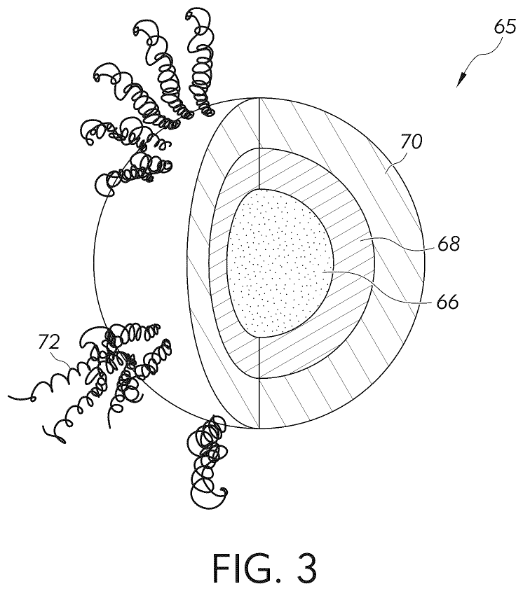

FIG. 3 depicts a partial cut-away perspective view of a magnetic nanoparticle 65 of the plurality of magnetic nanoparticles 18. A core 66 of each of the plurality of magnetic nanoparticles 18 may be made up of a ferromagnetic material such as, not limited to, iron oxide. Ferromagnetic material includes microscopic domains that may be oriented to become temporarily magnetic. Once the alternating magnetic field 26 ceases, the plurality of magnetic nanoparticles 18 will have no remnant magnetization. When the inductive magnetic field generator element 22 applies the alternating magnetic field 26 to the plurality of magnetic nanoparticles 18, the plurality of magnetic nanoparticles 18 align their microscopic domains to become magnetic and, in the process, induces hysteresis the plurality of magnetic nanoparticles 18. The elevation of temperature of the plurality of magnetic nanoparticles 18 due to the induced hysteresis also heats the surrounding tissue at the target site 12 in which the plurality of magnetic nanoparticles 18 have been placed.

In some embodiments, the core 66 of each of the plurality of magnetic nanoparticles 18 may be embedded with radioactive isotopes, such as Yttrium-90. Additionally or alternatively. each of the plurality of magnetic nanoparticles 18 comprises a first layer 68 that entirely covers the core 66. The first layer 68 may comprise a biocompatible coating, such as a polyethylene glycol coating as a non-limiting example, which may be released upon thermal activation. In embodiments, each of the plurality of magnetic nanoparticles 18 may include an iron oxide core 66 and a biocompatible coating disposed on the iron oxide core 66 as on via the first layer 68. Each of the plurality of magnetic nanoparticles 18 may include a radioactive isotope embedded in the biocompatible coating.

In embodiments, each of the plurality of magnetic nanoparticles 18 may include a therapeutic coating comprising one or more therapeutic agents. The one or more therapeutic agents may include a chemotherapeutic agent. In some embodiments, a second layer 70 entirely covers the first layer 68. The second layer 70 may include a therapeutic coating, such as, but not limited to, therapeutically relevant peptides and derivations of antibodies. The second layer 70 may be a drug-eluting surface for controlled release of chemotherapeutics, e.g., Cisplatin. Localized hyperthermia of even 2-3° C. has been shown to increase the efficacy of both radiation and chemotherapy treatments. A combination approach with specific targeting through the system 10 disclosed herein may be a viable option for some cancer patients and be an improvement over radiation or chemotherapy alone.

Furthermore, in some embodiments, the second layer 70 may include various organic or inorganic polymer coatings that may be chemically-modified to display functional groups, such as including targeting ligands 72. In embodiments, the plurality of magnetic nanoparticles 18 may include targeting ligands extending from the biocompatible coating. The plurality of magnetic nanoparticles 18 may have a variety of targeting moieties, depending on the intended cancer target.

If the plurality of magnetic nanoparticles 18 reach saturation magnetization, continued application of the alternating magnetic field 26 will not continue to increase the magnetism of the plurality of magnetic nanoparticles 18 such that temperature of the magnetic nanoparticles 18 will no longer increase.

Referring now to FIG. 4, the retention element 20 may include a transdermal patch 27. On one side of the transdermal patch 27 there is an adhesive layer (not shown from this perspective) that is configured to adhere to the patient 14. The operator of the disclosed system 10 adheres the retention element 20 on the outside of the patient 14 proximate the diseased tissue at the target site 12. The transdermal patch 27 may include the inductive magnetic field generator element 22, which is in the form of at least one flexible, yet, tightly-wound coil of copper wire configured to emit the alternating magnetic field 26 while receiving the electrical signal 24, such as an alternating current, from the signal generator 23. The inductive magnetic field generator element 22 may or may not be located within the retention element 20, depending on an embodiment. In some embodiments, the signal generator 23 is included in the console 30, and in other embodiments the signal generator is included within the retention element 20.

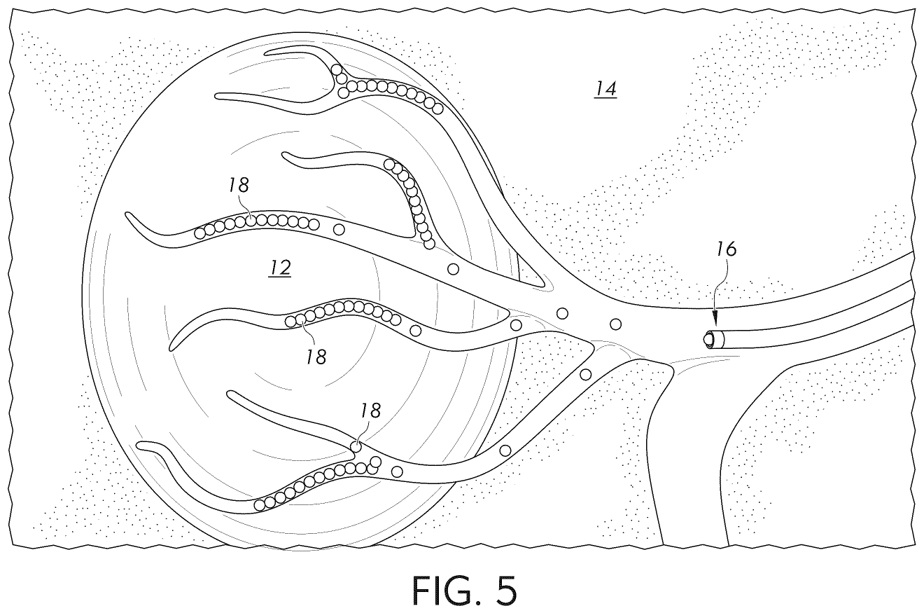

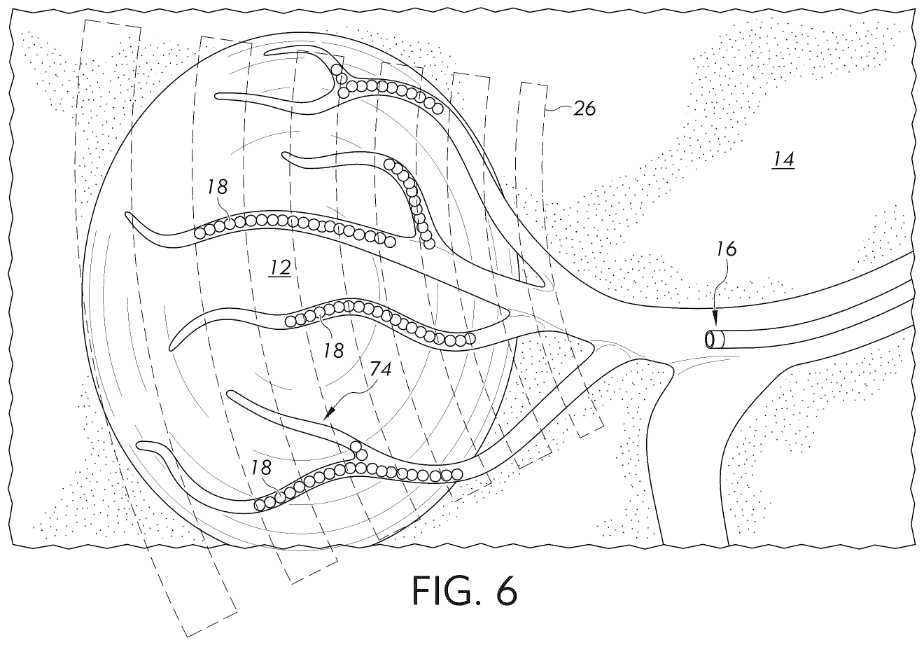

FIGS. 5 and 6 show schematic views of the delivery of the plurality of magnetic nanoparticles 18 into a plurality of vessels 74 (FIG. 6) of the patient 14, according to one or more embodiments shown and described herein. Referring to FIG. 5, a delivery probe 16, such as, and not limited to, a transfemoral or transradial catheter, is shown being steered through a vessel, such as the hepatic artery, to the target site 12 within the patient 14. As shown in FIG. 6, the delivery probe 16 is configured to deliver the plurality of magnetic nanoparticles 18 to a plurality of vessels 74. As the hepatic artery tends to supply blood preferentially to tumor sites while the healthy liver tissue is primarily fed by the portal vein, use of this hepatic artery by the delivery probe 16 would allow for delivery of the plurality of magnetic nanoparticles 18 to the target site 12 proximate the diseased tissue in the patient 14, sparing surrounding liver tissue from the hyperthermal ablation delivered via the present system 10. FIG. 6 shows an exemplary embodiment of the plurality of magnetic nanoparticles 18 distributed across multiple vessels 74 at the target site 12. FIG. 6 further shows the alternating magnetic field 26, produced by the inductive magnetic field generator element 22 and signal generator 23, interacting with the plurality of magnetic nanoparticles 18.

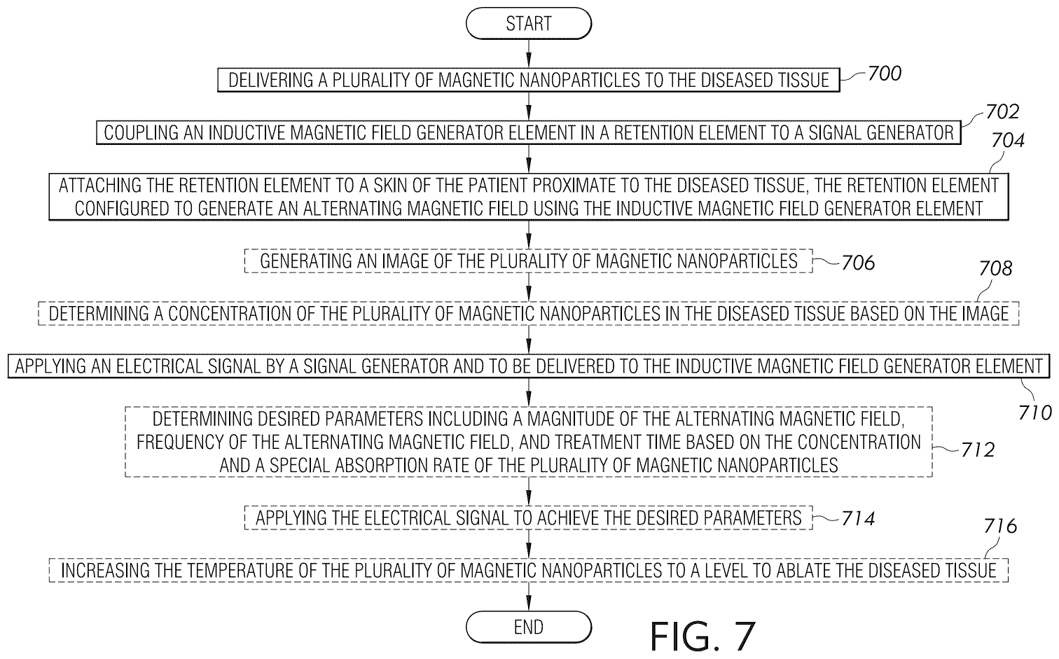

FIG. 7 shows a flowchart depicting a method for thermally ablating diseased tissue at a target site 12 in a patient 14. The method may include a greater or fewer number of steps in any order without departing from the scope of the present disclosure.

At block 700, a plurality of magnetic nanoparticles 18 are delivered to the diseased tissue at a target site 12. The diseased tissue being ablated may be within a liver. Moreover, the plurality of magnetic nanoparticles 18 may be delivered to diseased tissue at the target site 12 such as in the liver via a catheter disposed in a hepatic artery of the patient 14.

At block 702, an inductive magnetic field generator element 22 in the retention element 20 is coupled to the signal generator 23. At block 704, the retention element 20 including the inductive magnetic field generator element 22 disposed therein is attached to a skin of the patient 14 proximate to the diseased tissue. The retention element 20, as described herein, is configured to generate the alternating magnetic field 26 using the inductive magnetic field generator element 22 (and the signal generator 23). In embodiments, the inductive magnetic field generator element 22 may be attached to the skin of the patient 14 proximate the target site 12 without the retention element 20. As described above, the alternating magnetic field 26 interacts with the plurality of magnetic nanoparticles 18 to increase a temperature of the plurality of magnetic nanoparticles 18. In some embodiments of the method, the alternating magnetic field 26 increases the temperature of the temperature of the plurality of magnetic nanoparticles 18 to greater than or equal to 60° C.

In embodiments, at blocks 706-708, an image of the plurality of magnetic nanoparticles 18 may be generated as described herein to determine a concentration of the plurality of magnetic nanoparticles 18 in the diseased tissue based on the image. Determining the concentration of the plurality of magnetic nanoparticles 18 at the target site 12 may be conducted using imaging data produced by the imaging system 34 (FIG. 2). Additionally or alternatively, the concentration of the plurality of magnetic nanoparticles 18 at the target site 12 may be produced by the controller circuit 32 (FIG. 2) having data on a quantity of the nanoparticles delivered to the target site 12.

At block 710, the electrical signal 24 produced by the signal generator 23 is applied to be delivered to the inductive magnetic field generator element 22. The electrical signal 24 may be generated using the signal generator 23 that is coupled to the inductive magnetic field generator element 22.

In selected additional or alternative embodiments, the alternating magnetic field 26 is generated using the inductive magnetic field generator element 22 and the electrical signal 24, wherein the alternating magnetic field 26 interacts with the plurality of magnetic nanoparticles 18 to induce hysteresis. At blocks 712-714, the alternating magnetic field 26 is controlled by adjustment or application of a particular electrical signal 24. At block 712, a magnitude of the alternating magnetic field 26, a frequency of the alternating magnetic field 26, and a treatment duration is determined as desired parameters based on the concentration and a special absorption rate of the plurality of magnetic nanoparticles 18. At block 714, the electrical signal 24 is applied and/or adjusted to achieve the desired parameters as determined in block 712.

At block 716, the temperature of the plurality of magnetic nanoparticles 18 is increased based on the induced hysteresis to a level to ablate the diseased tissue (e.g., to a desired temperature to ablate the diseased tissue, which may be to the desired temperature of greater than or equal to 60° C. to ablate the diseased tissue). In embodiments, the temperature of the plurality of magnetic nanoparticles 18 and/or the diseased tissue at the target site 12 may be monitored via the detector 40.

Thus, the systems and methods disclosed herein may provide for a single, minimally invasive treatment that results in the ablation of all margins of a cancerous tumor, no matter the size, shape, or stage of the tumor, while also preserving healthy tissue. The visualization of the magnetic nanoparticles in combination with other factors, such as, the distance between the retention element and the target site, and the specific absorption rates associated with nanoparticles of certain size and material composition, may allow the user of the system to provide focused, accurate, and controlled hyperthermal ablation of cancerous cells to meet the needs of individual patients.

### ASPECTS LISTING

Aspect 1. A system for thermally ablating a diseased tissue at a target site in a patient includes a plurality of magnetic nanoparticles for disposal at the target site and a retention element configured to be attached to the patient proximate to the diseased tissue. The system includes an inductive magnetic field generator element disposed within the retention element and a signal generator coupled to the inductive magnetic field generator element. The signal generator is configured to generate an electrical signal. The inductive magnetic field generator element is configured to generate an alternating magnetic field based on the electrical signal, and the alternating magnetic field is configured to interact with the plurality of magnetic nanoparticles at the target site to induce hysteresis and thereby increase a temperature of the plurality of magnetic nanoparticles to a desired temperature to ablate the diseased tissue.

Aspect 2. The system of Aspect 1, wherein the retention element includes a patch comprising an adhesive layer configured to adhere to the patient proximate to the diseased tissue.

Aspect 3. The system of Aspect 1 or Aspect 2, wherein the alternating magnetic field may be configured to increase the temperature of the plurality of magnetic nanoparticles to greater than or equal to 60° C. to ablate the diseased tissue.

Aspect 4. The system of any of Aspect 1 to Aspect 3, wherein the system further includes an imaging system configured to generate an image containing a visualization of the plurality of magnetic nanoparticles, and a controller circuit is in communication with the signal generator and the imaging system. The controller circuit may be configured to estimate a concentration of the plurality of magnetic nanoparticles within the diseased tissue based on the image and identify a specific absorption rate of the plurality of magnetic nanoparticles.

Aspect 5. The system of Aspect 4, wherein the controller circuit is configured to determine an intensity of the electrical signal, a frequency of the electrical signal, and a treatment duration to apply based on the estimated concentration of the plurality of magnetic nanoparticles within the diseased tissue and the specific absorption rate of the plurality of magnetic nanoparticles.

Aspect 6. The system of any of Aspect 1 to Aspect 5, further including a detector configured to monitor the alternating magnetic field, the detector attached to the retention element.

Aspect 7. The system of any of Aspect 1 to Aspect 6, wherein each of the plurality of magnetic nanoparticles includes an iron oxide core and a biocompatible coating disposed on the iron oxide core.

Aspect 8. The system of Aspect 7, wherein the biocompatible coating may include a polyethylene glycol coating.

Aspect 9. The system of Aspect 7 or Aspect 8, wherein each of the plurality of magnetic nanoparticles may include a radioactive isotope embedded in the biocompatible coating.

Aspect 10. The system of any of Aspect 7 to Aspect 9, wherein the plurality of nanoparticles comprise targeting ligands extending from the biocompatible coating.

Aspect 11. The system of any of Aspect 1 to Aspect 10, wherein the plurality of magnetic nanoparticles further comprise a therapeutic coating that includes one or more therapeutic agents.

Aspect 12. The system of Aspect 11, wherein the one or more therapeutic agents comprise a chemotherapeutic agent.

Aspect 13. The system of any of Aspect 1 to Aspect 12, further including a controller circuit that is in communication with the signal generator. The controller circuit is configured to estimate a concentration of the plurality of magnetic nanoparticles within the diseased tissue, and the controller circuit is configured to identify a specific absorption rate of the plurality of magnetic nanoparticles. The system further includes a detector that is configured to monitor the electrical signal. The detector is configured to produce current level data associated with the electrical signal, and the detector is attached to the retention element and in communication with the controller circuit. The detector is configured to deliver the current level data to the controller circuit. The controller circuit is configured to determine and apply an intensity of the alternating magnetic field based on the current level data associated with the electrical signal, the estimated concentration of the plurality of magnetic nanoparticles within the diseased tissue, and the specific absorption rate of the plurality of magnetic nanoparticles.

Aspect 14. The system of Aspect 13, wherein the controller circuit is configured to determine and apply a treatment duration, the intensity of the alternating magnetic field, and a frequency of the alternating magnetic field based on the current level data, the concentration of the plurality of magnetic nanoparticles within the diseased tissue, and the specific absorption rate of the plurality of magnetic nanoparticles.

Aspect 15. The system of any of Aspect 1 to Aspect 14, further including a controller circuit that is in communication with the signal generator and configured to estimate a concentration of the plurality of magnetic nanoparticles within the diseased tissue, and the controller circuit is further configured to identify a specific absorption rate of the plurality of magnetic nanoparticles. The controller circuit is configured to determine and apply each of an intensity, a treatment duration, and a frequency of the alternating magnetic field to be delivered to the plurality of magnetic nanoparticles based on the concentration of the plurality of magnetic nanoparticles within the diseased tissue and the specific absorption rate of the plurality of magnetic nanoparticles.

Aspect 16. A system for thermally ablating diseased tissue at a target site in a patient may include a plurality of magnetic nanoparticles for disposal at the target site, and an inductive magnetic field generator element disposed proximate to the diseased tissue. The system further includes a signal generator coupled to the inductive magnetic field generator element and configured to generate an electrical signal. The inductive magnetic field generator element is configured to generate an alternating magnetic field based on the electrical signal, the alternating magnetic field configured to interact with the plurality of magnetic nanoparticles at the target site to induce hysteresis and thereby increase a temperature of the plurality of magnetic nanoparticles to a desired temperature to ablate the diseased tissue. The system also includes a detector that is configured to monitor the alternating magnetic field, and the detector is configured to be coupled to the inductive magnetic field generator element.

Aspect 17. The system of Aspect 16, wherein the inductive magnetic field generator element comprises a copper wire coil.

Aspect 18. The system of Aspect 16 or Aspect 17, further comprising an imaging system configured to generate an image of the plurality of magnetic nanoparticles and a controller circuit. The controller circuit is configured to: estimate a concentration of the plurality of magnetic nanoparticles based on the image; estimate a specific absorption rate of the plurality of magnetic nanoparticles; and determine and apply an intensity of the alternating magnetic field, a frequency of the alternating magnetic field, and a treatment duration, wherein the controller circuit is configured to generate the alternating magnetic field based on the concentration of the plurality of magnetic nanoparticles at the target site and the specific absorption rate associated with the plurality of magnetic nanoparticles.

Aspect 19. The system of Aspect 16 or Aspect 17, the system further includes an imaging system configured to generate an image of the plurality of magnetic nanoparticles.

Aspect 20. The system of any of Aspect 16 to Aspect 19, wherein the inductive magnetic field generator element and the detector may be disposed in a patch comprising an adhesive layer configured to be attached to the patient proximate to the diseased tissue.

Aspect 21. The system of any of Aspect 16 to Aspect 20, wherein each of the plurality of magnetic nanoparticles includes an iron oxide core and a biocompatible coating disposed on the iron oxide core.

Aspect 22. The system of Aspect 21, wherein the biocompatible coating may include a polyethylene glycol coating.

Aspect 23. The system of any of Aspect 16 to Aspect 22, wherein each of the plurality of magnetic nanoparticles further includes a therapeutic coating comprising one or more therapeutic agents.

Aspect 24. The system of any of Aspect 21 to Aspect 23, wherein each of the plurality of magnetic nanoparticles further includes a radioactive isotope embedded in the biocompatible coating.

Aspect 25. The system of any of Aspect 16 to Aspect 24, further including a controller circuit in communication with the signal generator, configured to estimate a concentration of the plurality of magnetic nanoparticles within the diseased tissue, and configured to calculate an estimated specific absorption rate of the plurality of magnetic nanoparticles. Also, the controller circuit may be configured to control the delivery of the alternating magnetic field as a desired alternating magnetic field by determining field parameters including an intensity of the alternating magnetic field for delivery, a frequency of the alternating magnetic field for delivery, and a treatment duration based on the estimated concentration of the plurality of magnetic nanoparticles within the diseased tissue and the estimated specific absorption rate associated with the plurality of magnetic nanoparticles, determining an electrical signal based on the field parameters, and applying the electrical signal to achieve the desired alternating magnetic field.

Aspect 26. A method for thermally ablating diseased tissue at a target site in a patient includes delivering a plurality of magnetic nanoparticles to the diseased tissue at the target site; attaching a retention element including an inductive magnetic field generator element disposed therein to a skin of the patient proximate to the diseased tissue; generating an electrical signal using a signal generator coupled to the inductive magnetic field generator element; generating an alternating magnetic field using the inductive magnetic field generator element and the electrical signal, wherein the alternating magnetic field interacts with the plurality of magnetic nanoparticles to induce hysteresis; and increasing a temperature of the plurality of magnetic nanoparticles based on the induced hysteresis to a desired temperature to ablate the diseased tissue.

Aspect 27. The method of Aspect 26, wherein the alternating magnetic field increases the temperature of the plurality of magnetic nanoparticles to the desired temperature of greater than or equal to 60° C. to ablate the diseased tissue.

Aspect 28. The method of Aspect 26 or Aspect 27, further including generating an image of the plurality of magnetic nanoparticles and determining a concentration of the plurality of magnetic nanoparticles in the diseased tissue based on the image.

Aspect 29. The method of any of Aspect 26 to Aspect 28, further including selecting the electrical signal to be produced by a signal generator based on: estimating a concentration of the plurality of magnetic nanoparticles within the diseased tissue, selecting a specific compositional material and average size of the plurality of magnetic nanoparticles, determining the distance between the inductive magnetic field generator element and the plurality of magnetic nanoparticles delivered to the diseased tissue, and/or determining the temperature of the plurality of magnetic nanoparticles.

Aspect 30. The method of any of Aspect 26 to Aspect 29, further including determining a set of field parameters including a magnitude of the alternating magnetic field, a frequency of the alternating magnetic field, and a treatment duration, and applying the electrical signal based on the set of field parameters.

Aspect 31. The method of any of Aspect 26 to Aspect 30, further including determining the temperature of the diseased tissue and/or determining the temperature of the plurality of magnetic nanoparticles.

Aspect 32. The method of any of Aspect 26 to Aspect 31, wherein the diseased tissue may be liver tissue, and the plurality of magnetic nanoparticles are delivered to the liver tissue via a catheter disposed in a hepatic artery of the patient.

While embodiments of the present disclosure have been described with respect to at least one embodiment, the present disclosure can be further modified within the spirit and scope of this disclosure. This application is therefore intended to cover any variations, uses, or adaptations of the present disclosure using its general principles. Further, this application is intended to cover such departures from the present disclosure as come within known or customary practice in the art to which the present disclosure pertains and which fall within the limits of the appended claims.

1. A system for thermally ablating a diseased tissue at a target site in a patient, the system comprising:
a plurality of magnetic nanoparticles for disposal at the target site;
a retention element configured to be attached to the patient proximate to the diseased tissue;
an inductive magnetic field generator element disposed within the retention element; and
a signal generator coupled to the inductive magnetic field generator element and configured to generate an electrical signal, wherein the inductive magnetic field generator element is configured to generate an alternating magnetic field based on the electrical signal, and wherein the alternating magnetic field is configured to interact with the plurality of magnetic nanoparticles at the target site to induce hysteresis and thereby increase a temperature of the plurality of magnetic nanoparticles to a desired temperature to ablate the diseased tissue.

2. The system of claim 1, wherein the retention element comprises a patch comprising an adhesive layer configured to adhere to the patient proximate to the diseased tissue.

3. The system of claim 1, wherein the alternating magnetic field is configured to increase the temperature of the plurality of magnetic nanoparticles to the desired temperature of greater than or equal to 60° C. to ablate the diseased tissue.

4. The system of claim 1, further comprising an imaging system configured to generate an image containing a visualization of the plurality of magnetic nanoparticles, and a controller circuit in communication with the signal generator and the imaging system, the controller circuit being configured to estimate a concentration of the plurality of magnetic nanoparticles within the diseased tissue based on the image and identify a specific absorption rate of the plurality of magnetic nanoparticles.

5. The system of claim 4, wherein the controller circuit is configured to determine an intensity of the alternating magnetic field, a frequency of the alternating magnetic field, and a treatment duration to apply based on the estimated concentration of the plurality of magnetic nanoparticles within the diseased tissue and the specific absorption rate of the plurality of magnetic nanoparticles.

6. The system of claim 1, further comprising a detector configured to monitor the alternating magnetic field, the detector attached to the retention element.

7. The system of claim 1, wherein each of the plurality of magnetic nanoparticles comprises an iron oxide core and a biocompatible coating disposed on the iron oxide core.

8. The system of claim 7, wherein the biocompatible coating comprises a polyethylene glycol coating.

9. The system of claim 7, wherein each of the plurality of magnetic nanoparticles comprise a radioactive isotope embedded in the biocompatible coating.

10. The system of claim 7, wherein the plurality of magnetic nanoparticles comprise targeting ligands extending from the biocompatible coating.

11. The system of claim 1, wherein each of the plurality of magnetic nanoparticles further comprise a therapeutic coating comprising one or more therapeutic agents.

12. The system of claim 11, wherein the one or more therapeutic agents comprise a chemotherapeutic agent.

13. The system of claim 1 further comprising:
a controller circuit in communication with the signal generator, the controller circuit configured to estimate a concentration of the plurality of magnetic nanoparticles within the diseased tissue, the controller circuit configured to identify a specific absorption rate of the plurality of magnetic nanoparticles;
a detector configured to monitor the electrical signal, the detector configured to produce current level data associated with the electrical signal, the detector attached to the retention element and in communication with the controller circuit, the detector configured to deliver the current level data to the controller circuit.
wherein the controller circuit is configured to determine and apply an intensity of the alternating magnetic field based on the current level data associated with the electrical signal, the concentration of the plurality of magnetic nanoparticles within the diseased tissue, and the specific absorption rate of the plurality of magnetic nanoparticles.

14. The system of claim 13 wherein the controller circuit is configured to determine and apply a treatment duration, the intensity of the alternating magnetic field, and a frequency of the alternating magnetic field based on the current level data, the concentration of the plurality of magnetic nanoparticles within the diseased tissue, and the specific absorption rate of the plurality of magnetic nanoparticles.

15. The system of claim 1 further comprising:
a controller circuit in communication with the signal generator, the controller circuit configured to estimate a concentration of the plurality of magnetic nanoparticles within the diseased tissue, the controller circuit configured to identify a specific absorption rate of the plurality of magnetic nanoparticles,
wherein the controller circuit is configured to determine and apply each of an intensity, a treatment duration, and a frequency of the alternating magnetic field to be delivered to the plurality of magnetic nanoparticles based on the concentration of the plurality of magnetic nanoparticles within the diseased tissue and the specific absorption rate of the plurality of magnetic nanoparticles.

16. A system for thermally ablating diseased tissue at a target site in a patient, the system comprising:
a plurality of magnetic nanoparticles for disposal at the target site;
an inductive magnetic field generator element disposed proximate to the diseased tissue;
a signal generator coupled to the inductive magnetic field generator element and configured to generate an electrical signal, wherein the inductive magnetic field generator element is configured to generate an alternating magnetic field based on the electrical signal, and wherein the alternating magnetic field is configured to interact with the plurality of magnetic nanoparticles at the target site to induce hysteresis and thereby increase a temperature of the plurality of magnetic nanoparticles to a desired temperature to ablate the diseased tissue; and
a detector configured to monitor the alternating magnetic field, the detector configured to be coupled to the inductive magnetic field generator element.

17. The system of claim 16, wherein the inductive magnetic field generator element comprises a copper wire coil.

18. The system of claim 16, further comprising an imaging system configured to generate an image of the plurality of magnetic nanoparticles and a controller circuit configured to:
estimate a concentration of the plurality of magnetic nanoparticles based on the image;
estimate a specific absorption rate of the plurality of magnetic nanoparticles; and
determine and apply an intensity of the alternating magnetic field, a frequency of the alternating magnetic field, and a treatment duration, wherein the controller circuit is configured to generate the alternating magnetic field based on the concentration of the plurality of magnetic nanoparticles at the target site and the specific absorption rate associated with the plurality of magnetic nanoparticles.

19. A method for thermally ablating diseased tissue at a target site in a patient, the method comprising:
delivering a plurality of magnetic nanoparticles to the diseased tissue at the target site;
attaching a retention element including an inductive magnetic field generator element disposed therein to a skin of the patient proximate to the diseased tissue;
generating an electrical signal using a signal generator coupled to the inductive magnetic field generator element;
generating an alternating magnetic field using the inductive magnetic field generator element and the electrical signal, wherein the alternating magnetic field interacts with the plurality of magnetic nanoparticles to induce hysteresis; and
increasing a temperature of the plurality of magnetic nanoparticles based on the induced hysteresis to a desired temperature to ablate the diseased tissue.

20. The method of claim 19, wherein the alternating magnetic field increases the temperature of the plurality of magnetic nanoparticles to the desired temperature of greater than or equal to 60° C. to ablate the diseased tissue.
